(19) United States
(12) Patent Application Publication
(10) Pub. No.: US 2019/0390373 A1
(43) Pub. Date: Dec. 26, 2019

(54) FIBERS WITH SEGMENTS, THEIR PREPARATION AND APPLICATIONS THEREOF

(71) Applicant: ASSOCIATION FOR THE ADVANCEMENT OF TISSUE ENGINEERING AND CELL BASED TECHNOLOGIES & THERAPIES (A4TEC, Braga (PT)

(72) Inventors: Luca GASPERINI, Braga (PT); Alexandra Margarida PINTO MARQUES, Porto (PT); Rui Luis GONÇALVES DOS REIS, Porto (PT)

(21) Appl. No.: 16/395,335

(22) Filed: Apr. 26, 2019

Related U.S. Application Data
(63) Continuation of application No. PCT/IB2017/056654, filed on Oct. 26, 2017.

(30) Foreign Application Priority Data
Oct. 26, 2016 (PT) 109697

(51) Int. Cl. D01F 8/18; D01F 9/04; D01F 1/10; A61L 26/00

(52) U.S. Cl. CPC D01F 8/18; D01F 9/04; D01F 1/10; C08L 5/04; A61L 26/008; A61L 26/0066; A61L 26/0052

(57) ABSTRACT

The present application discloses a method to produce fibers with different segments along the major axis. The different segments can be constituted of the same materials that entrap different objects (molecules, particles) or can be made of different materials. The different segments are made thanks to a junction and by alternating the dispensing of such materials using different inlet channels. This method allows the production of fibers that can be used as processed or after being further manipulated by other processes, as single devices or as building blocks to construct devices that are more complex. Fibers with different segments along the axis can be exploited for a wide range of applications as medical devices and/or as drug delivery system and/or as matrices to be used for acellular tissue regeneration and cellular tissue engineering/regenerative medicine strategies and/or as supports for imaging in high throughput screening.

### CROSS-REFERENCE TO RELATED PATENT APPLICATIONS

This application is a continuation application of International Patent Application No. PCT/IB2017/056654, filed Oct. 26, 2017, which claims the benefit of priority to Portuguese Patent Application number PT 109697 filed Oct. 26, 2016, both of which are incorporated by reference as if set forth in their respective entireties herein.

### TECHNICAL FIELD

This application relates to a process to produce fibers with segments and their applications. In particular, the present invention relates to a novel method capable of producing continuous fibers made of different segments along the major axis of the fiber. Fibers are used as processed or as building blocks to produce products that hold potential application as medical devices and/or as drug delivery system and/or as matrices to be used for acellular tissue regeneration and cellular tissue engineering/regenerative medicine strategies.

### SUMMARY

The present application is related to a method to produce fibers with different segments along the major axis comprising the following steps:
selection of at least two fluids to fill the same number of reservoirs respectively, connected to the inlet channels;
application of pressure to at least two reservoirs containing different fluids;
the fluids under pressure flow into an inlet channel up to a junction point;
application of pressure to one of the reservoirs while the other reservoirs enter their inactive phase;
the pressured fluid flows into an outlet channel;
application of pressure to another reservoir while the other reservoirs enter their inactive phase;
the fluid under pressure flows into the inlet channel pushing forward the previous fluid into the outlet channel; steps 4 to 7 are repeated forming a fluid column with different segments until the desired fiber length is obtained;

In one embodiment the pressure applied to the reservoir in the active phase stops when the reservoir enters the inactive phase.

In another embodiment the fluid are solutions of a polymeric precursor that harden to form a solid.

In yet another embodiment the fluids comprise different molecules, particles, cells or small parts of living tissues.

In one embodiment the fluids are thermoresponsive fluids that undergo thermal hardening by means of temperature variation.

In one embodiment the fluid column is chemically hardened.

In another embodiment the hardening chemicals are salt solutions in a concentration between 50 mM and 5 M.

In one embodiment the fluid column is hardened by means of incident UV light.

In another embodiment the fiber is hardened inside or outside the outlet channel.

In yet another embodiment the minimum time to apply pressure is 100 ms.

In one embodiment the size of the channels ranges between 10 μm and 5 mm in width.

The present application also relates to fibers with different segments along the major axis obtained by the method described previously.

In one embodiment the segments of the fiber are constituted by different materials, wherein the materials are the fluid solutions in their solid state.

In one embodiment the segments are constituted by the same materials.

In another embodiment the segments vary in width and length.

In yet another embodiment at least one segment comprises a gradient of molecules, particles, cells or small parts of living tissues.

In one embodiment the fibers are used to produce multi-component medical devices.

### BACKGROUND ART

Most of the processes aimed at producing multi-component fibers provide a way to obtain such fibers with the component recognizable in the cross sectional area. In the present invention, the components are recognizable along the major axis of the fiber while along the cross-sectional area the component is the same.

Patent WO2010127119 A2 (Multiphasic microfibers for spatially guided cell growth) discloses multiphasic microfibers and in particular the methods of producing these fibers as scaffolds for promoting spatially guided cell growth and proliferation. All references show microfibers with different phases recognizable in the cross section of the fibers. The invention disclosed herein is aimed at producing separate segments along the major axis of the fiber while the cross section of the fiber can be homogenous. Furthermore, the invention disclosed herein has broader applications and is not limited to spatially guiding cell growth in one single or more phases of the fibers. Our invention is not limited to electrospraying/electrospinning the fluid, but is focused on the methods of producing fibers with different segments along its axis.

Similarly, patent WO9921507 (Synthetic Fibres for medical use and method of making the same) discloses a method of producing fibers and the references and drawings refer to different components recognizable in the cross sectional area while the present application refers to separate segments along the major axis of the fiber while the cross section of the fiber at each segment is homogenous and made of the same component.

Similarly, patent US20070232169 (Medical devices containing multi-component fibers) discloses a method of producing fibers and references and drawing refers to different components recognizable in the cross sectional area while the present application refers to separate segments along the major axis of the fiber while the cross section of the fiber at each segment is homogenous and made of the same material.

Patent WO0147567A2 (fibers providing controlled active agent delivery) refers to methods of producing multicomponent fibers. These methods refer explicitly to fibers made with a core-shell (or more layers) structures and as such to multicomponent fibers with the different elements recognizable in the cross sectional area (FIG. 2, 3). The present application refers to separate segments along the major axis of the fiber while the cross section of the fiber at each segment is homogenous and made of the same component.

Patent WO2014143866 (Core-Sheath fibers and method of making and using same) refers to methods of producing multicomponent fibers. These methods refer explicitly to fibers made with a core-shell (or more layers) structures and as such to multicomponent fibers with the different elements recognizable in the cross sectional area. The present application refers to separate segments along the major axis of the fiber while the cross section of the fiber at each segment is homogenous and made of the same component.

Patent WO2004061173 A2 (Multicomponent fiber incorporating thermoset and thermoplastic polymers) discloses multicomponent fiber containing at least two polymer components arranged in distinct zones or segments across the cross-section of the fiber wherein at least one component of the fiber contains a thermoplastic polymer and at least one component of the fiber contains a thermoset polymer. The present application has a general approach and is not limited to specific materials but can be applied to a wide range of fluids. Furthermore, the present disclosure is aimed at producing separate sections along the major axis of the fiber while the cross section of the fiber at each segment is homogenous and made of the same component.

Similarly, U.S. Pat. No. 7,045,211 B2 (Crimped thermoplastic multicomponent fiber and fiber webs and method of making) describes a process that provides a crimped multicomponent fiber containing at least two polymer components arranged in a crimpable configuration in distinct zones or segments across the cross-section of the fiber wherein one component comprises a dielectrically susceptible material. The invention that we propose does not relate to specific materials or particular configuration of these materials. The process proposed in the present application is mainly aimed at keeping the axial components of the fibers separated.

U.S. Pat. No. 6,162,382 (A Process of making multicomponent fiber) describes Multicomponent fibers and methods of producing the same are provided such that an inter-domain boundary layer is interposed between distinct domains formed of incompatible polymers to minimize (if not eliminate entirely) separation of the domains at their interfacial boundary. In the present application the process does not rely on incompatible polymers but the components can be made of compatible polymers (for example the same polymer but loaded with different drugs). Furthermore, there is no explicit need or presence of inter-domain boundary layers and the separation between the axial segments is obtained by the process itself and the control of its parameters.

U.S. Pat. No. 7,737,060 B2 (Medical devices containing multi-component fibers) discloses medical devices containing multi-component fibers defining a “multi-component polymeric fiber” as a polymeric fiber within whose cross-section can be found at least two distinct cross-sectional components, each of different composition, for instance, (a) because the polymer content varies between the components, or (b) where one or more therapeutic agents are provided, because the therapeutic agent content varies between the components, or (c) because both the polymeric and therapeutic agent content varies between the components, among numerous other possibilities. In the present invention, the multi-component fibers have the same component in the cross-sectional area thus forming fibers that do not necessarily have at least two distinct cross-sectional components. In addition, in the case of the present invention the polymeric content or the therapeutic agents varies in the different section but the sections are not in the cross-sectional area. One cross sectional area can be entirely made of one polymer and another cross-sectional area entirely made of a different polymer or containing different therapeutic agents. The technology disclosed herein is based on the ability to obtain fibers with different segments along the major axis and to obtain medical devices with such fibers.

Patent WO2005037336 A1 (Hydrogel-containing medical articles and methods of using and making the same) describes an invention that relates generally to medical articles comprising a high-water content hydrogel made by crosslinking a protein with activated polyethylene glycols. The medical articles may further include an active agent, such as an agent that confers antimicrobial, analgesic, and/or wound healing activities to the hydrogel. The invention further provides methods for treating a wound using the medical articles described. The invention however does not disclose the process of making multicomponent fibers through a junction that results in fibers containing different components along the major axis. The key point of the device itself claimed in the present application is the ability to design a patch containing the desired components in the desired place of the patch.

Patent CA 2523620 C (Drug releasing biodegradable fiber for delivery of therapeutics) generally refers to a method of manufacturing a fiber comprising, dissolving a biodegradable polymer in a first solvent to form a polymer solution, wherein said first solvent is poorly miscible in water. In the technology disclosed herein, there is no limitation in the miscibility of the polymer, nor its degradability.

Patent WO 1997002811 A1 (Hydrogel patch) discloses a patch that is composed of a polymeric material which forms a gel with water. Furthermore, it discloses a patch made of hydrogels, which contain components that enhance the performance of the gel for a particular purpose. The process disclosed in the present invention may be applied to polymeric precursors other than hydrogel precursors. The present application relates to a process to produce fibers containing different segments along the major axis and the devices made with these fibers.

### BRIEF DESCRIPTION

The present application provides a method to produce fibers with different segments along the major axis. The fibers are produced starting by at least two fluids (or suspensions) flowing into a channel that are connected through a junction. Furthermore, this application provides a method of producing devices with such fibers.

The term device refers to patches, bandage, cellular and acellular matrices for tissue engineering, imagining and high throughput screening, controlled drug release.

To make the fibers, a fluid is placed in a reservoir connected to a channel (e.g. a tube). When the reservoir is pressurized, the fluid will flow into the channel. The channel is connected to a junction together with the other channels that make the system. The junction can be a simple T or Y tubing junction or can have a more advanced geometry able to connect more channels together such as but not limited to a microfluidic chip and a polymeric mold. The junction is made in such way that the fluids coming from the different channels can join in at least one common outlet channel. By applying pressure on at least one channel, only the fluids from those channels will flow through the outlet. By stopping the application of pressure on those channels, the flow will stop. By pressurizing other channels, new and different fluids will flow in the outlet channel pushing forward the fluid already present in the outlet channel. By repeating this process, the outlet channel is filled by different fluids without mixing or with minimal mixing so that different segment along the path of the outlet channel can be identified. After this, the fluid is hardened so that the segments can remain separated. The exiting fluid is hardened in or outside the outlet channel and in different ways based on its nature. It is hardened by a temperature variation, by the presence of a chemical or any external stimuli.

Fibers have attracted a lot of interest in the biomedical field given their high surface to volume ratio and relatively easiness of production. Fibers are commonly made by extrusion of a polymeric solution that is then hardened following different strategies. With the process of the present application and the use of a junction, it is possible to make fibers with different segments of different composition along the major axis. These segments can have different sizes. The width of the segments depends on the nature of the starting solution, the size of the outlet channel of the junction and the parameters of the process such as but not limited to the overall flow rate at the outlet channel. The width of the fiber is mainly influenced by the width of the channel where the fluid that forms the fibers flows. The width is also influenced by the nature of the fluid. The length of the segments only depends on the parameters of the process. The parameters are adjusted to obtain segments of the desired length. This process allows the production of fibers that can be used as they are or as building blocks to construct more advanced devices.

Any processing parameter that increases the flow rate of the fluid will increase the length of the segment formed by that fluid. The parameters that influence the length of the segments are the amount of pressure applied and the duration of the said applied pressure. This method allows the production of fibers that can be used as processed or after being further manipulated by other processes, as single devices or as building blocks to construct devices that are more complex.

Fibers with different segments along the major axis can be exploited for a wide range of applications as medical devices and/or as drug delivery system and/or as scaffolds to be used in acellular tissue regeneration and cellular tissue engineering/regenerative medicine strategies.

The advantage given by this technology is the use of different components in different segments so that with a single fiber a larger range of complementary strategies can be followed in comparison to fibers made of one single component. For example, one small segment along the fiber may be made of a material and another segment may be made of other material. The different materials may entrap the same molecule but being the materials different in nature the release profile of that molecule will be different. Considering another example two segments may be made of the same material but with different molecules entrapped to obtain a controlled spatial release of the different molecules. The fiber may be used to entrap cells in at least one segments. This will allow obtaining cylindrical segments containing cells in the fiber. The cylindrical shape compared to the spherical one has the advantage to increase the external surface and as such increase the diffusion of nutrients/oxygen and removal of metabolites. By combining cellular and acellular segments of different materials, the acellular segment can be degraded disconnecting the cellular segments from the fibers in a selective mode.

Commercially available patches do not address successfully and completely all the aspects of wound healing. In fact, when the skin is injured, the body initiates a cascade of processes (hemostasis, inflammation, proliferation, and remodeling) ultimately leading to wound re-epithelialization and re-establishment of the skin's barrier function. For a wound to heal successfully, all four phases must occur in the proper sequence and period. The healing of an adult skin wound is a complex process requiring the collaborative efforts of different tissues and cell lineages. Differently from the commercially available patches that are typically made of one single component, the devices, for example patches, made with the fibers produced with the process of the present application are multi component. The single fibers containing different segments can be aligned side by side. It will be possible to design the patch so that selected drugs, growth factors, and materials comprised in the different segments will be in close contact with the different microenvironments of the wound addressing different needs and selectively aiding to the collaborative effort required for successful wound healing. Furthermore, because these patches are made with a bottom-up approach they will have more advantages. The patch can be produced in different sizes so that the best patch for different wound extensions and healing phases can be manufactured. Small parts of the patch can act as sensors without interfering with its efficacy so that important parameters for wound healing such as pH or oxygenation, through the presence of lactate, can be monitored. The patches made with the fibers with the process disclosed in the present application may also contain viable cells. This great advantage could drastically increase the efficacy and range of application of the patch. The presence of selected cell lineages in the patch could optimize the healing process given the ability of cells to monitor external cues and respond with the production of bioactive molecules. In addition, this will allow the patch to be effectively used for other important applications than wound healing. For example, for therapies involving the transplant of allogeneic endocrine tissue.

The fibers can also be used for the screening of cell/material interaction. The segment of the fiber containing cells can be observed and analyzed for a specific cell response. Since the segments containing cells are embedded in the fiber it is possible to track them based on the position along the fiber. (segment 1 is the first segment, segment 3 is the third segment along the fiber and so on). As the composition of those segments change along the fiber, based on the position of such segment, is possible to determine the original composition.

### BRIEF DESCRIPTION OF THE DRAWINGS

The features of the present technology are explained in detail in the appended claims. The invention itself may be best understood by reference to the following detailed description that describes exemplary embodiments of the present technology. Without intent to limit the disclosure herein, the invention, its benefits, and advantages may be best understood by reference to the accompanying drawings.

FIGS. 1A-1B: Show schematics of the apparatus needed for the current invention. The apparatus itself is not claimed by this invention but only the process to make fibers with different segments.

FIGS. 2A-2C: Show schematics and simplified representations of the process. Two channel (called inlet 1 and inlet 2) join through a junction to a common outlet channel. By applying pressure, a fluid flows into the inlet channel. By selectively and alternatively applying pressure to the inlet channels different segments are formed, one made of solution 1 (coming from channel 1) and one made of solution 2 (coming from channel 2).

FIGS. 3A-3B: Show schemes showing the outcome of the process and the definition of the following terms “Major axis of fiber, width of segment, length of segment”.

FIG. 4: Shows a flow chart of the operational procedure to make the fiber.

FIG. 5: Shows fibers made of two components (black and white) where the black component has an increasing size.

FIG. 6: Shows fibers made with the same method of FIG. 5 but this time the white component has an increasing size while the black component is constant.

FIG. 7: Shows fibers made of three components, White (W), Blue (B), and Red (R).

FIG. 8: Shows a continuous fiber with polylactic acid particles entrapped in some segments.

FIG. 9: Shows the segments containing the particles of FIG. 8 release from the rest of the fiber.

FIG. 10: Shows a segment of the fiber containing living cells.

FIG. 11: Shows a scheme to produce a patch for wound healing applications as an example.

FIGS. 12A-12C: Show some of the steps to produce a 2 components patch.

FIGS. 13A-13C: Show three different patches of different sizes and number of components.

FIG. 14: Shows a fiber with segments of different length containing living cells.

FIG. 15: Shows a fiber with segments comprised of a gradient of different cells compositions.

### DETAILED DESCRIPTION OF THE EMBODIMENTS

Reference will now be made to the attached figures to describe the present invention. The detailed description and technical contents of the present application will be disclosed herein according to a preferable embodiment. The embodiments are not used to limit its execution scope.

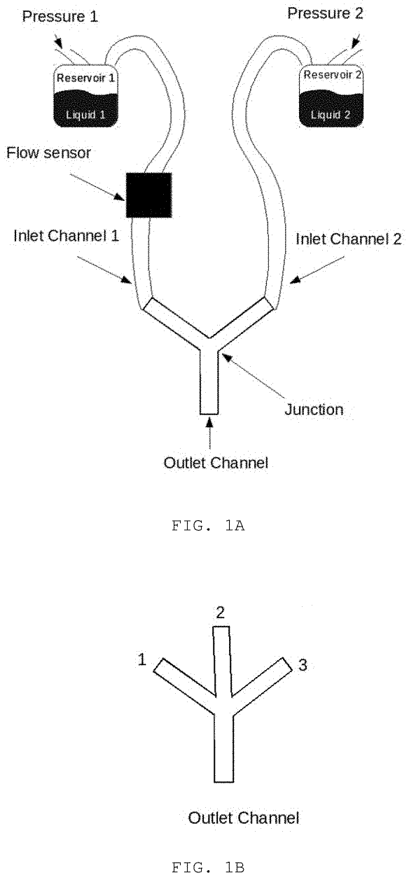

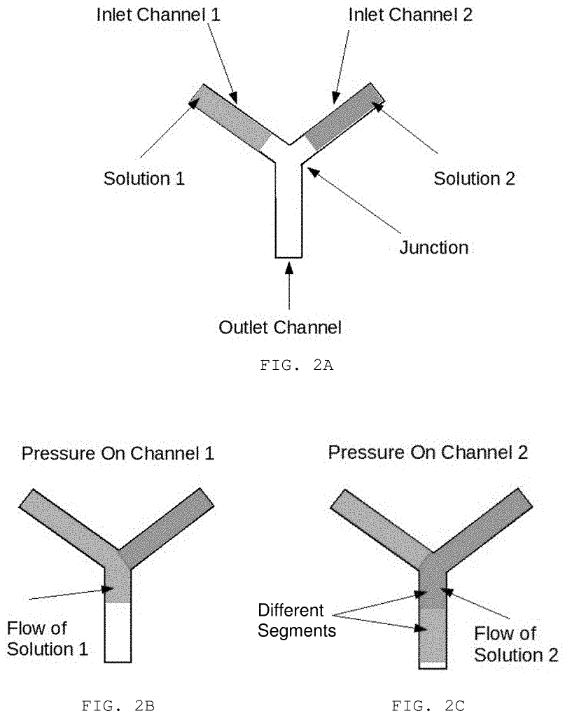

In one embodiment, two inlet channels are used in the production of the fibers (FIG. 1A). However, more channels can be used and the scheme can be modified accordingly (e.g. in FIG. 1B, where 1, 2 and 3 are inlet).

In another embodiment, there is a reservoir for each channel (FIG. 1A), the reservoir contains a fluid. The fluid is a solution of a polymeric precursor that can harden and forms a solid. The reservoir is connected to a source of pressure and to the inlet channel. When pressure is applied to the reservoir, the fluid in the reservoir flows into the channel up to a junction. The junction is such that the fluids coming from the different reservoirs are connected. FIG. 1A shows the junction for two inlet channels while FIG. 1B shows the junction for three inlets. In another embodiment the apparatus contains a flow sensor able to measure the flow of the fluids inside the channels and control the flow rate of the fluid by a feedback system to the pressure source. Furthermore, the flow sensor is placed along one or more channels in any place suitable for the application.

FIGS. 2A-2C show one way of carrying out the invention using two inlet channels.

In one embodiment, pressure is applied to both reservoirs so that the fluids flow into the inlet channel up to the junction point (FIG. 2A). At this point, the pressure on both reservoirs is stopped. Pressure is then applied to only one of the reservoirs (e.g FIG. 1A—Reservoir 1) so that the fluid contained (Fluid 1) flows into the outlet channel (FIG. 2B). For this purpose, a minimal pressure is applied to reservoir 2 (e.g. FIG. 1A —Reservoir 2) to keep the flow rate of fluid 2 at 0 and avoid re-fluxes into Inlet Channel 2. This pressure ranges from 0.1 to 5% of the pressure on reservoir 1. Then, pressure on Reservoir 1 is stopped (and eventually also on Reservoir 2 if pressure to Reservoir 2 was applied to avoid re-fluxes). With references to FIG. 2C, pressure is applied on Reservoir 2 so that Fluid 2 flows into the outlet channel. (Similarly for this purpose some pressure may be applied to Reservoir 1 to keep the flow rate of fluid 1 at 0 and avoid refluxes into Inlet channel 1). This way fluid 2 will push forward fluid 1 into the outlet channel without mixing or with minimal mixing forming in fact a column of fluid with two different segments. Then, pressure on reservoir 2 is stopped (and eventually also on reservoir 1 if pressure to reservoir 1 was applied to avoid re-fluxes). Then the process showed in FIG. 2B and FIG. 2C can be repeated to create more segments.

In another embodiment, one or more channels have the pressure applied continuously if the final column of fluid with different segments can still be obtained.

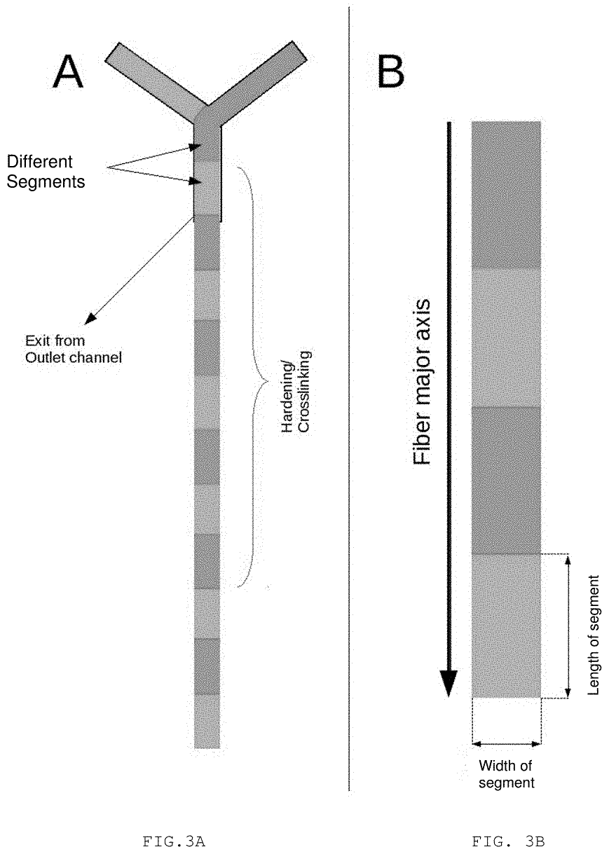

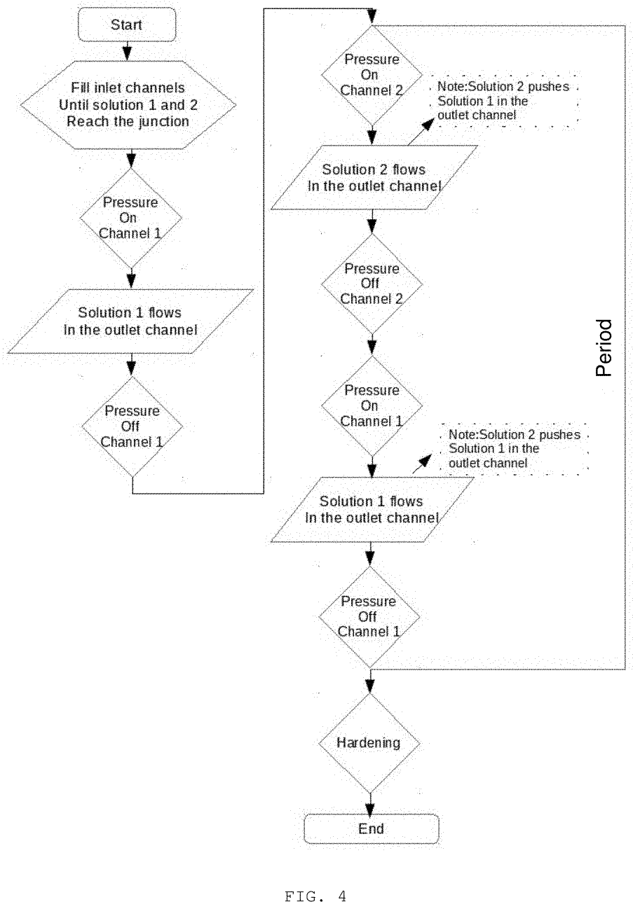

FIG. 3A shows the steps after the formation of the column of fluid with different segments. In one embodiment, the fluid column is hardened so that a fiber with the mechanical properties of a solid is formed. This way the segments remain separate. The fiber is formed inside or outside the outlet channel. The fiber is formed by different crosslinking/hardening strategies based on the nature of the starting solution. In one embodiment, the hardening is achieved by temperature variation if the fluid is thermoresponsive. In this case, the column of fluid flows in an environment with a temperature gradient to form a solid fiber.

In another embodiment, the hardening is achieved chemically and the column of fluid flows in an environment where chemical crosslinkers are present to form a solid fiber. Chemical crosslinkers are those suitable for the fluid used to produce the fibers. When the solution is alginate the chemical used is a water solution of Calcium chloride and or Barium chloride at a concentration ranging from 50 mM to 5 M. When gellan gum solution is used the hardening bath is a salt solution (sodium chloride) in a concentration ranging from 50 mM to 5 M.

In another embodiment the hardening occurs by means of an electromagnetic stimulus. In this case, the column of fluid is subject by the external stimulus (e.g. light) to form the fiber.

In another embodiment a fiber is produced using a junction made by channels of 190 μm in diameter. The fiber is made of two segments that correspond to two different solutions contained in the reservoir. One solution is an alginate solution at 1.5% w/vol in water while the other is a gellan gum solution at 0.75% w/vol in 0.25M Sucrose. The fiber is produced by alternating the dispensing of the two fluids. The gellan gum segments is produced by applying a pressure of 80000 PA for 2.75 s while the alginate part is produced by applying a pressure of 150000 Pa for 0.25 s. The fiber produced is 400 μm in diameter. The gellan gum segments is 3 mm long and the alginate segment is 15 mm long.

In another embodiment a fiber is produced using a junction made by channels of 190 μm in diameter. The fiber is made of two segments of the same materials that correspond to two different solutions contained in the reservoir. One solution is an alginate solution at 2% w/vol in water while the other is an alginate solution at 2% w/vol phosphate buffer (PBS). The fiber is produced by alternating the dispensing of the two fluids. The alginate in PBS segment is produced by applying a pressure of 15000 Pa for 2.5 s while the alginate part is produced by applying a pressure of 200000 Pa for 0.5 s. The fiber produced is 400 μm in diameter. The alginate in PBS segments is 2.5 mm long and the alginate in water segment is 10 mm long.

In one embodiment the fiber is produced with segments whose material composition changes along the fiber. The fiber is produced using a junction made by channels of 190 μm in diameter. The fiber is made of two segments, one obtained from the alginate solution contained in the reservoir (alginate solution at 1.5% w/vol in water) and the other segment formed by a solution produced by mixing two other solutions at different ratios.

The fiber is produced by alternating the dispensing of the two fluids (the alginate and the solution produced after the mixing).

FIG. 3B defines some terms used in the present application such as “fiber major axis, width, and length of segment”. The “Fiber major axis” corresponds to the length of the fiber or, in other terms, the line passing through the center of the fiber section whose direction is the one of the flow in the outlet channel. The width of the segment corresponds to the diameter of the fiber or the width of the section of the fiber in case of a not-round fiber. The length of the segment corresponds to the distance between two adjacent segments along the major axis of the fiber.

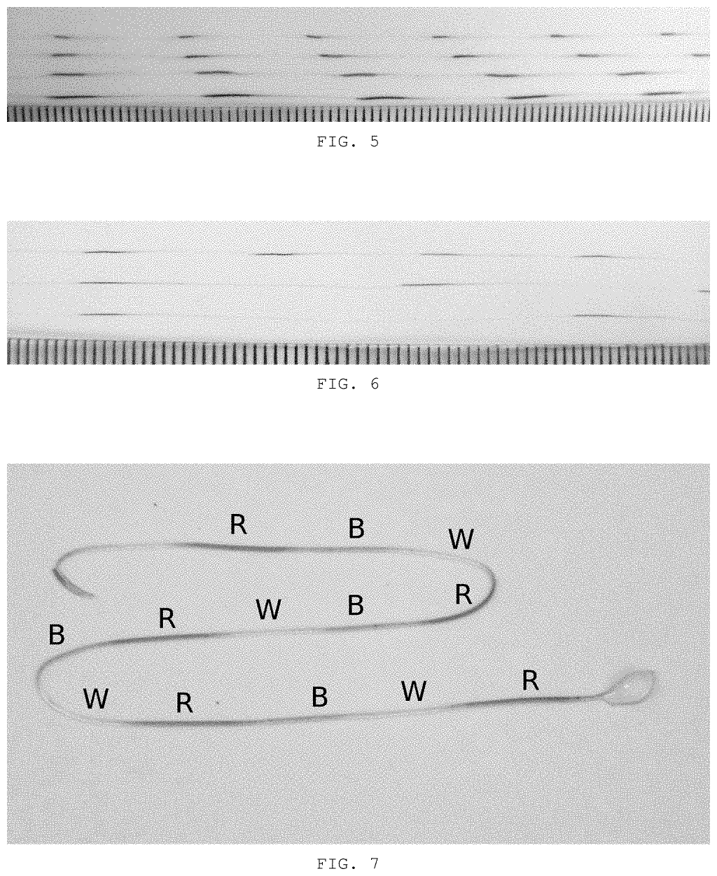

The time needed to perform the stages of the diagram of FIG. 4, is referred as a “Period”.

An active phase is to be understood as the phase when the highest amount of pressure is applied to a reservoir, and there is flow of solution in the inlet channel, in the junction and in the outlet channel. The technical elements of reservoirs, or channels in this phase, are also considered “active” in this phase.

On the other hand, an inactive phase is to be understood as the phase when the lowest amount of pressure, considered residual pressure, is applied to a reservoir, and there is no flow of solution in the inlet channel connected with that reservoir. The technical elements of reservoirs, or channels in this phase, are also considered “inactive” in this phase.

The possibility to promote a fast change between active phase and inactive phase, by switching the pressure levels applied in the reservoirs, allows producing a fiber in a short time.

In one embodiment the pressure applied to the reservoir is equal to or higher than 1000 Pa, depending on the solutions used.

In one embodiment the relation between the flow rate in the channels and the pressure applied to the reservoirs depends on the nature of the solution.

In one embodiment the minimum time of application of pressure to the reservoirs is 100 ms.

In one embodiment the size of the channels ranges between 10 μm and 5 mm in width and from 1 to 1000 mm in length.

In one embodiment the pressure of the “inactive reservoirs” is higher than zero to avoid backflow because of the fluid flowing from the active channel. The pressure of the inactive channels varies with the nature of the fluid used and the geometry of the channels. Such pressure varies from 0.1 to 5% of the pressure on the active reservoir.

### Hardening Methods:

### Hardening Outside the Outlet Channel:

In one embodiment the solutions that form the fiber are hardened outside the outlet channel by means of being in contact with salt solutions.

In one embodiment the polymeric solution that hardens to form a gel solution is gellan gum at 1% weight/volume that hardens forming a solid when placed in contact with a salt solution.

In one embodiment the polymeric solution that hardens to form a gel solution is alginate at 2% weight/volume that hardens forming a solid when placed in contact with a salt solution containing divalent cations such as Calcium or Barium.

In one embodiment the solution is made of anionic polymers such as carrageenan, alginate, gellan gum that hardens upon contact with salts. For Gellan gum monovalent cations of the salt electrically shield the negatively charged group allowing a tighter aggregation of the polymer forming a solid For Alginate divalent or trivalent cations, in addition to their electrical screening effect, bind together different negative groups also forming solids. The concentration of salts (Sodium Chloride, Calcium Chloride, barium Chloride) in solution can vary between 20 mM to 5 M. For these polymers the solution in the outlet channel containing the segments is immersed in the salt solution to form a solid fiber.

Hardening Inside the Outlet Channel with Temperature:

In one embodiment the solutions that form the segments of the fiber are hardened inside the outlet channel by a change in temperature. In another embodiment, the solution is made of a thermoresponsive polymer that is a polymer (collagen, gelatin) that forms a solid upon temperature variation. For these polymers, the solution in the outlet channel containing the segments undergoes a temperature change to form a solid. The temperature change depends on the nature of the solution, it may be between 4° C. (solution) to 37° C. (formation of a solid) for collagen at 0.4% weight/volume or 45° C. (solution) to 25° C. (formation of a solid) for gelatin at 15% weight/volume.

In another embodiment the solution is a collagen solution with the reservoir at a temperature of 4° C. that hardens forming a solid when temperature changes to 37° C.

Hardening Inside the Outlet Channel with UV Light:

In one embodiment the solutions that form the segments of the fiber comprise photoinitiators and the fibres are hardened inside the outlet channel by means of exposure to UV light.

In another embodiment the solution is made of polymers that can form a solid when irradiated by UV light (250-500 nm) in the presence of a suitable photoinitiator (such as Irgacure). The polymers may be in the family of the methacrylated or acrylated polymers (modified hyaluronic acid, modified gellan gum, modified gelatin with acrylate or methacrylate groups) carrying a double bond on the polymeric backbone. When irradiated by light the photoinitiator is activated and a chemical reaction is started that breaks those double bonds and forms new bonds between the polymeric chains and forming a solid. For these polymers the solution in the outlet channel containing the segments is irradiated by UV light to form a solid fiber.

In another embodiment the solution is methacrylated or acrylated gellan gum at 2% weight/volume with 2-Hydroxy-4′-(2-hydroxyethoxy)-2-methylpropiophenon as photoinitiator at 0.05% weight/volume and the reservoir is kept in the dark, the solution can then harden when placed under UV radiation (250-500 nm wave length) for a period of time necessary for the formation of a solid (from 2 seconds to 5 minutes).

### EXAMPLES

### Example 1.1

FIG. 5 shows fibers with segments of different sizes. A flow sensor is used to control the flow rate of fluid 1 (dark in the figure, gellan gum 1% in 0.25M Sucrose solution). Fluid 2 consists of a solution of alginate 1.5% in water. Each step of the rules placed as reference on the bottom of the picture is 1 mm. The fibers in the picture is made using the following parameters:

Period: 3 seconds;
Time of application of pressure 1: 2.55 seconds;
Time of application of pressure 2: 0.45 seconds;
Flow rate related to pressure 1: from top to bottom 2.5, 5, 10, 20 μL/min;
A pressure 2 of 7500 Pa was used to avoid re-fluxes while pressure 2 was in its inactive phase;
Size of inlet channels=size of outlet channels=190×390 μm;
Diameter of fiber produced: 400 μm;

In these conditions the relation between pressure applied and flowrate is as follow:

### For Gellan gum 1%:

| Flow Rate (μL/min) | Pressure 1 (Pa) |
|---|---|
| 2.5 | 50000 |
| 5 | 71200 |
| 10 | 89200 |
| 20 | 105000 |

For alginate 1.5%

| Flow Rate (μL/min) | Pressure 2 (Pa) |
|---|---|
| 223 | 200000 |

### Example 1.1.1

When different lengths of the segments in the fiber are needed, other parameters can be used. A higher flow rate relates with longer segments both for the gellan gum and for alginate segments.

### For Gellan gum 1%

| Flow Rate (μL/min) | Pressure 1 (Pa) |
|---|---|
| 0.6 | 25000 |
| 6.2 | 75000 |
| 7.5 | 82000 |
| 15 | 95000 |

For alginate 1.5%

| Flow Rate (μL/min) | Pressure 2 (Pa) |
|---|---|
| 2.5 | 4600 |
| 5 | 8000 |
| 7.5 | 11300 |
| 10 | 15000 |
| 15 | 23000 |
| 15.3 | 25000 |
| 33.5 | 50000 |
| 53.5 | 75000 |
| 91 | 100000 |
| 151 | 150000 |

The length of the segments relates to the flow rate (or pressure) as follows:

### Length=Volume/Section Area

Volume=FlowRate×time of application of pressure
Section Area=(πD\hat{ }2)/4 with D the diameter of the fiber produced.

The solution coming from the outlet channel is hardened using a hardening bath composed of a solution of 100 mM Calcium Chloride in water.

### Example 1.2

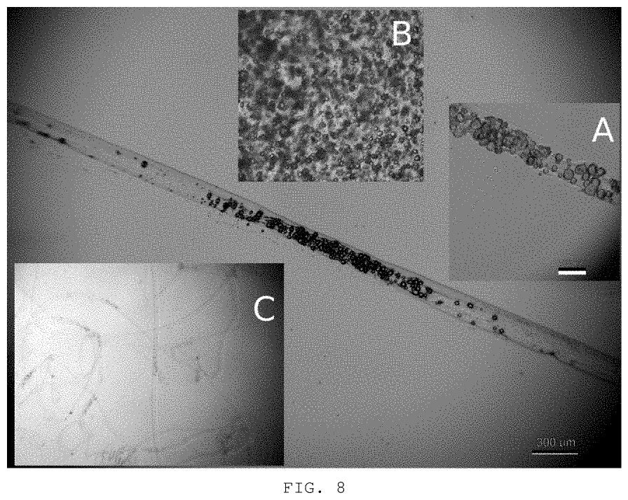

FIG. 6 shows fibers similar to FIG. 5 but made with different parameters. Each step of the ruler placed as reference on the bottom of the picture is 1 mm. The segments made by fluid 1 (dark) are of the same size but the distance is changed. From top to bottom:

| | | Time | | | Length | Length |
|---|---|---|---|---|---|---|
| | | of | | | of | of |
| | | flow | | Time of | segment | segment |
| Flowrate | Pressure | of | Pressure | application | made of | made of |
| of fluid 1 | of fluid | fluid | of fluid | of pressure | fluid 1 | fluid 2 |
| (uL/min) | 1 (Pa) | 1 (s) | 2 (PA) | 2 (s) | (mm) | (mm) |
| 7.5 | 82000 | 2.55 | 200000 | 0.45 | 2.5 | 12 |
| 10 | 89200 | 1.95 | 200000 | 1.05 | 2.5 | 26 |
| 5.5 | 72000 | 3.5 | 200000 | 1.5 | 2.5 | 42 |

A pressure 2 of 7500 Pa was used to avoid re-fluxes while reservoir 2 was in its inactive phase.

Size of inlet channels=size of outlet channels=190×390 μm
Diameter of fiber: 400 μm

### Example 1.3

FIG. 7 shows a fiber made of three components (Gellan gum 1%) with different dyes representing different loaded molecules. The fiber was made with a junction composed of three inlets and 1 outlet of size 190×390 μm. Each inlet channel was connected to a reservoir containing a gellan gum solution at 1% in 0.25M sucrose of different color (red, blue and white→R, B, W respectively)) and the fiber was made by alternating the flow of the three fluids into the outlet channel. The flowrate of the three fluids was 10 uL/min for 2.55 seconds which correspond to an applied pressure of 89200 Pa. The solution coming from the outlet channel was hardened using a hardening bath composed of a solution of 100 mM Calcium Chloride in water.

### Example 1.4

Similarly, to example 1.3 the fiber is made of three components (collagen). The solutions were made from a starting collagen solution ranging from 0.05 to 0.4% in 0.2% acetic acid. To this solution was added 10% in volume of concentrate and the pH was adjusted to pH 7 with the addition of NaOH. The system (reservoir, inlet channels, junction) is kept at 4° C. while the outlet channel was placed at 37° C. for hardening.

### Example 1.5

Similarly, to example 1.3 the fiber is made with three components (gelatin). The solutions were made from a starting gelatin solution ranging from 1 to 20% in water. The system (reservoir, inlet channels, junction) is kept at 39° C. while the outlet channel was placed at 4° C. for hardening.

### Example 1.6

Similarly, to example 1.3 the fiber was made with three components (methacrylated gellan gum). The solutions were made from a starting methacrylated gellan gum solution ranging from 0.5 to 4% in water. The system (reservoir, inlet channels, junction) is kept in the dark while the outlet channel was placed under UV light for hardening for a period of time that ranges from 1 second to 10 minutes.

### Example 1.7

Similarly, to example 1.3 the fiber is made with two components. One component was produced from starting solution of gellan gum at 1% in water. The flowrate of the gellan gum was 10 uL/min for 2.55 seconds which correspond to an applied pressure of 89200 Pa. The second component was produced by mixing at different ratios two gellan gum solution at 1% in 0.25M Sucrose containing different cell lineages in suspension, mesenchymal/stromal stem cells and endothelial cells.

### Example 1.8

Similarly, to example 1.3 the fiber was made with two components. One component was produced starting from a solution of gellan gum at 1% in water. The flowrate of the gellan gum was 10 uL/min for 2.55 seconds which correspond to an applied pressure of 89200 Pa. The second component was produced by mixing two different solutions, one of pure gellan gum at 1% in 0.25M Sucrose the other of 0.5% hyaluronic acid and 1% gellan gum in 0.25M sucrose.

### Example 1.9

Similarly, to example 1.3 the fiber was made with two components. One component was produced from a starting solution of gellan gum at 1% in water. The flowrate of the gellan gum was 10 uL/min for 2.55 seconds which correspond to an applied pressure of 89200 Pa. The second component is produced by mixing two different solutions, one of pure gellan gum at 1% in 0.25M Sucrose the other of gellan gum at 1% and 0.5% chondroitin sulfate in 0.25M sucrose.

### Example 2.1

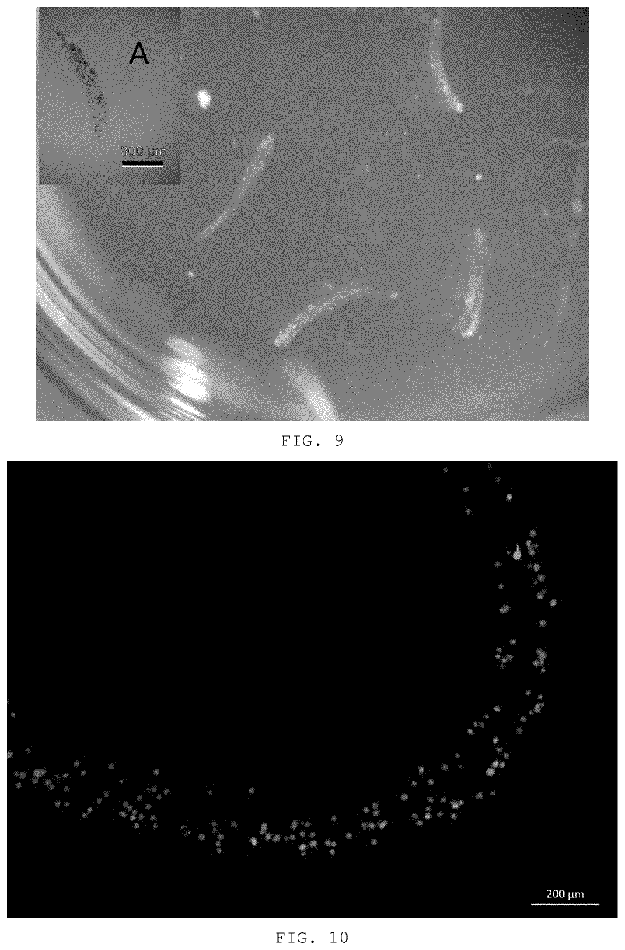

FIG. 8 shows solid particles entrapped in a segment of the fiber. Polylactic acid microparticles were mixed in a solution of gellan gum at 1% to obtain a suspension of particles in the polymeric solution (FIG. 8, inset B). A two inlet junction was used to obtain a continuous fiber of alginate containing segments of gellan gum loaded with micro particles (FIG. 8, inset C). The segments containing the particles was fabricated using a flowrate of 10 uL/min for 2.55 seconds which correspond to an applied pressure of 89200 Pa (while FIG. 8, Inset A shows the magnification of the segment containing the entrapped particles).

In one embodiments the particles are loaded with drugs and the fiber is used to provide sustained drug release for medical use.

### Example 2.2

FIG. 9 shows the segment containing the particles removed from the fiber. The initial fiber was made by two distinct materials namely a gellan gum (1%) containing Polylactic acid microparticles and alginate 2%.

After production the fiber was placed in a solution of alginase (10 U/ml) and left overnight. One unit (U) is defined as the quantity that results in an increase the absorption at 235 nm of 1.0 per minute per mL of sodium alginate solution at pH 6.3 and 37° C. The alginase selectively degrade the alginate portion of the fiber effectively releasing from the structure intact segments made of gellan gum. FIG. 9, Inset A shows the segment using a different illumination so that the particles entrapped are clearly visible.

### Example 3.1

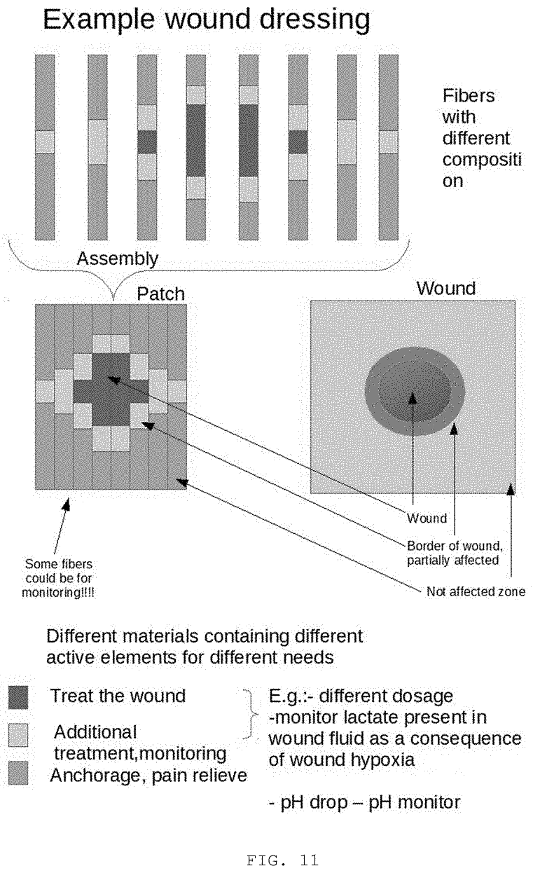

FIG. 10 shows a fluorescent picture of living cells entrapped in one segment of the fibers. A gellan gum solution in 0.25M sucrose was used to suspend 1 million human adipose stem cells for each ml of gellan gum. This suspension was used to form hydrogel segments inside a fiber made from alginate 1.5% weight/vol in water. The fiber was incubated in standard culture condition for 24 h. After this the fiber was stained with calcein (a molecule that once metabolized by living cells gives green fluorescence) and propidium iodide (a molecule that can stain the nuclei of dead cells). In one embodiment these fibers are used as a support for cells and the fiber is used for tissue engineering and regenerative medicine. In one embodiment cells are glucose responsive cells and the fiber is used for the release of insulin and the treatment of diabetes.

### Example 4.1

FIG. 11 shows a scheme to produce hydrogel patches using the fibers produced by this invention as building blocks. By assembling fibers made of segments of different materials and with different molecules in suspension or in solution a patch was made. The fibers are aligned side by side to reconstruct the desired geometry and distribution of materials and molecules on the patch. This way the patch is designed to be placed, for example, in a wound and target differently the different microenvironments that constitute the wound. Furthermore, one or more fibers or segment can contain monitoring molecules that monitor the environment of the wound. In one embodiment, a dual component fiber is inserted to monitor the pH of the wound and the presence of lactate.

### Example 4.2

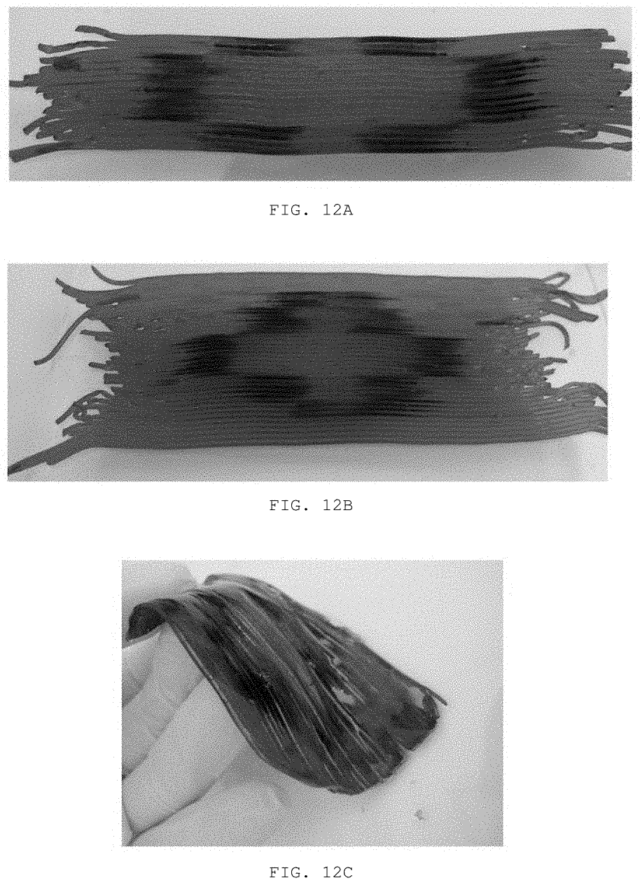

FIGS. 12A-12C show the successive steps to form the patch. The fibers (the building blocks of the patch) made of different segments are aligned side by side so that the desired design of the patch is obtained. FIG. 12 A shows the alignment of fibers made of two components. The design was obtained by changing the size of the segments and aligning the fibers accordingly. FIG. 12 B shows the addition of the fibers of 1 component to obtain the final bundle of fibers. The bundle of fiber is then coated with a solution of 10% gelatin at 37° C. to glue the fibers together. After leaving it at room temperature the gelatin hardens increasing the mechanical properties of the bundle and allow the fiber to be handled (FIG. 12 C).

### Example 4.3

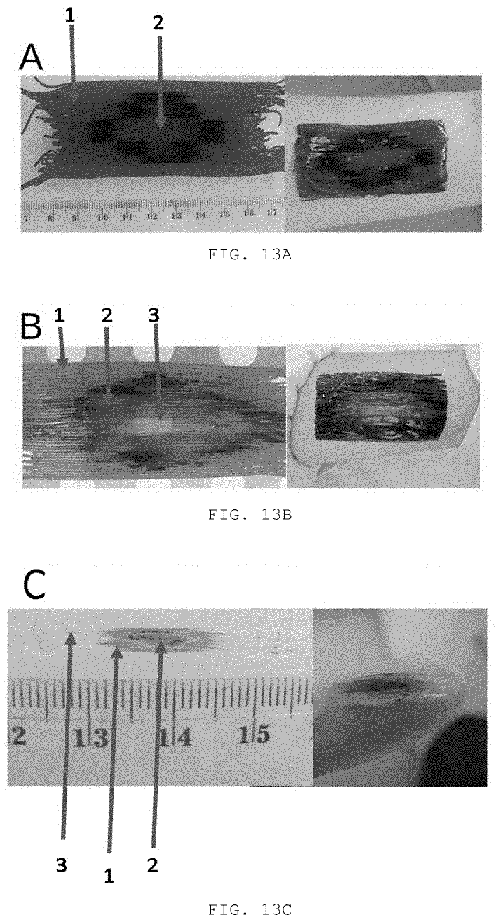

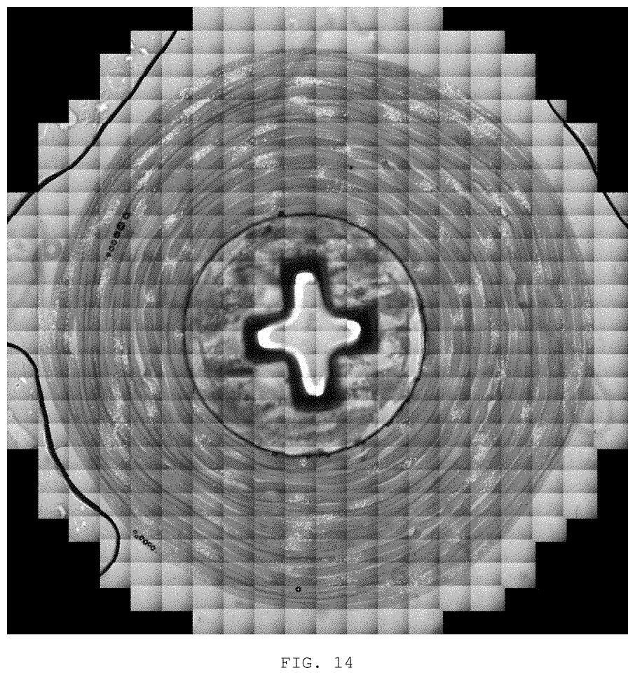

FIGS. 13A-13C show the patches. Segment 1 was made of a solution of gellan gum at 1% in water mixed with a dye (Alizating red) acting as a molecule. Segment 2 was made of a solution of gellan gum at 1% in water mixed with a dye (Alcyan blue) acting as a molecule. Segment 3 (transparent) is a solution of gellan gum at 1% in water. The left part the figure shows the patches before the coating while the right part shows the coated patches placed on a part of the. FIG. 13 A shows a wrist size patch made of two different components by bundling fibers made of components 1 and 2. FIG. 13 B shows a wrist size patch made of three different components by bundling fibers made of components 1, 2 and 3. FIG. 13 C shows a finger-tip size patch made of three different components by bundling fibers made of components 1, 2 and 3.

### Example 5.1

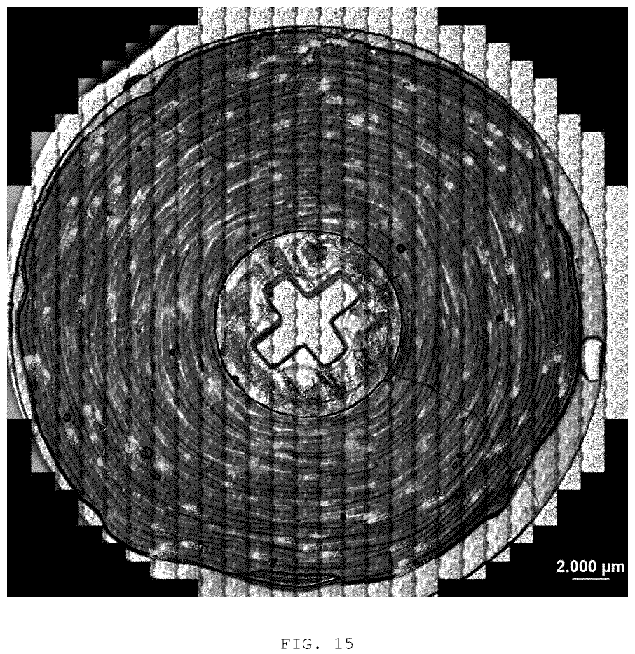

FIG. 14 shows a fiber rolled in a spool and placed under the microscope for image analysis. The fiber was made of two gellan gum solutions at 1% in 0.25M sucrose, one with cells in suspension and the other one without cells. The solution with cells contains 1 million human adipose stem cells for each ml of gellan gum that were stained with a fluorescent green (Calcein) for observation using a fluorescent microscope. The fiber was made in 100 seconds. The segments containing cells were made by linearly increasing the flow rate from 0 μL/min to 50 μL/min (from 0 Pa to 121000 Pa) in 100 seconds. The segments without cells were made by applying a pressure following a sinusoidal function with a period of 1 second and an amplitude of 150000 Pa.

The final fiber has segments of different length (constant width of 400 μm) containing cells. The smaller segments are in the center of the picture (close to the center of the spool marked with a X) while the longer segments are on the peripheral part of the picture. The segments produced range from 0 to 3 mm in length.

### Example 5.2

FIG. 15 shows a fiber rolled in a spool and placed under the microscope for image analysis. The fiber is made of three gellan gum solutions at 1% in 0.25M sucrose, two with cells in suspension and the other without cells. The solutions with cells contain 1 million human adipose stem cells for each ml of gellan gum that were stained with a fluorescent dye. Cells from one solution were stained in green and the other were stained in red (Calcein) for observation using a fluorescent microscope. The solutions containing cells were mixed with a microfluidic mixer to create fluid containing a gradient of red and green cells. The solution was mixed starting from the solution containing green cells flowing at 5 μL/min (71200 Pa). Then the flowrate was linearly decreased to 0 in 120 seconds. During the same 120 seconds period the flowrate of the solution containing red cells was increased from 0 to 5 uL/min. The solution coming from the mixer was then used to create the segments of the fiber containing cells. The segments of the fiber without cells were made by applying a pressure of 150000 Pa for 0.6 seconds over a period of 3 seconds.

1. An extruded segmented polymeric fiber comprising:
a plurality of different segments along a major axis of the polymeric fiber, including at least:
a first polymeric segment, and
a second polymeric segment, wherein the first and second polymeric segments each comprise polymeric material;
wherein the polymeric material of the first polymeric segment is different from the polymeric material of the second polymeric segment or,
wherein the first polymeric segment and the second polymeric segment comprise the same polymeric material with a different component, wherein said component is at least one of following elements: molecules, drugs, therapeutic agent, bioactive factors, growth factors, cells, particles, small parts of living tissues or a combination thereof.

2. The fiber according to claim 1, wherein the first and the second polymeric segments are connected.

3. The fiber according to claim 1, wherein the polymeric material is a hydrogel.

4. The fiber according to claim 1, wherein the first and second polymeric segments have different lengths and wherein the different length of each of the first and second polymeric segments is obtainable by applying different pressures.

5. The fiber according to claim 1, wherein the polymeric material of the first and second polymeric segments is selected from the group consisting of: polymeric precursor, anionic polymer, and thermoresponsive polymer.

6. The fiber according to claim 1, wherein the polymeric material of the first and second polymeric segments is selected from the group consisting of: gellan gum, alginate, collagen, gelatin, and carrageenan.

7. The fiber according to claim 1, wherein at least one polymeric segment comprises a concentration gradient of said component.

8. The fiber according to claim 6, wherein the gellan gum is a methacrylated gellan gum or a acrylated gellan.

9. The fiber according to claim 6, wherein the gelatin is a methacrylated gelatin or a acrylated gelatin.

10. The fiber according to claim 1, wherein the first polymeric segment comprises alginate and the second polymeric segment comprises gellan gum, or the first polymeric segment comprises gelatin and the second polymeric segment comprises collagen.

11. The fiber according to claim 1, wherein the fiber is 400 μm in diameter.

12. The fiber according to claim 1, wherein the polymeric segments vary in width and length.

13. (canceled)

14. (canceled)

15. (canceled)

16. (canceled)

17. A method for treating a patient using a patch comprising the extruded segmented polymeric fiber of claim 1 for wound healing.

18. A method of producing an extruded segmented polymeric fiber comprising different segments along the major axis, comprising:
preparing a first reservoir of a first polymeric solution;
preparing a second reservoir of a second polymeric solution;
connecting the first reservoir to a first inlet channel;
connecting the second reservoir to a second inlet channel;
connecting the first inlet channel and the second inlet channel to a junction;
connecting an outlet channel to the junction;
applying pressure to the first reservoir to push the first polymeric solution through the first inlet channel towards the junction;
applying pressure to the first reservoir to push the first polymeric solution and extrude the first polymeric solution into the outlet channel;
reducing the pressure of the first reservoir to push the first polymeric solution through the first inlet channel towards the junction, maintain the first polymeric solution at an edge of the junction;
applying pressure to the second reservoir to push the second polymeric solution through the second inlet channel towards the junction;
applying pressure to the second reservoir to push the second polymeric solution and extrude the second polymeric solution into the outlet channel;
reducing the pressure of the second reservoir to push the second polymeric solution through the second inlet channel towards the junction, maintain the second polymeric solution at the edge of the junction;
alternating the pressure applied to the first reservoir and the second reservoir to alternate the polymeric solution being extruded into the outlet channel;
stopping the pressure applied to the first reservoir and the second reservoir when the desired length of extruded fiber has been achieved.

19. The method according to claim 18, further comprising hardening the extruded fiber inside the outlet channel or outside the outlet channel.

20. The method according to claim 19, wherein the hardening of the extruded polymeric fiber is done by: temperature variation, chemical cross-linking, contact with a salt solution, irradiation by ultraviolet light in the presence of a suitable photo-initiator, or electromagnetic stimuli.

21. The method according to claim 18, wherein the inlet and outlet channels are connected to at least one flow sensor to measure the flow of the polymeric solutions.

22. The method according to claim 18, wherein the size of the channels ranges between 10 μm and 5 mm.

23. The method according to claim 18, wherein the pressure applied to the reservoirs is equal to or higher than 1000 Pa.

24. The method according to claim 18, wherein the pressure of one of the reservoirs range from 0.1% to 5% of the pressure of the other reservoir.
